(19) United States
(12) Patent Application Publication
(10) Pub. No.: US 2025/0027309 A1
(43) Pub. Date: Jan. 23, 2025

(54) CHLORINE DIOXIDE BIOFILM PREVENTION DEVICE

(71) Applicant: Kohler Co., Kohler, WI (US)

(72) Inventor: Yunjie Zhang, Orlando, FL (US)

(21) Appl. No.: 18/763,034

(22) Filed: Jul. 3, 2024

Related U.S. Application Data
(60) Provisional application No. 63/527,677, filed on Jul. 19, 2023.

(51) Int. Cl. E03D 9/03; A61L 2/18

(52) U.S. Cl. CPC E03D 9/03; A61L 2/18; A61L 2202/15

(57) ABSTRACT

Described herein is a biofilm prevention device including a first chamber configured to store a dry precursor, a second chamber configured to receive a predetermined volume of water, a first valve disposed between the first chamber and the second chamber configured to convey a predetermined quantity of dry precursor into the second chamber, and a second valve disposed at an outlet of the second chamber, the second valve configured to control a flow of chlorine dioxide solution to the plumbing fixture.

### CROSS-REFERENCE TO RELATED APPLICATION

This application claims priority benefit of Provisional Application No. 63/527,677 (Docket No. 010222-23015A) filed on Jul. 19, 2023, which is hereby incorporated by reference in its entirety.

### FIELD

The present application relates generally to biofilm prevention in and disinfection of plumbing fixtures. More specifically, the present disclosure relates to the application of a chlorine dioxide solution to plumbing fixtures for disinfection and to prevent biofilm growth.

### BACKGROUND

The presence of biofilm in plumbing fixtures not only has negative health implications, but also negatively impacts performance of the plumbing fixture. Specifically, biofilm build up may accumulate reducing flow area through the plumbing fixture. Additionally, biofilm may cause or accelerate clogging of the plumbing fixture by grabbing onto hair or other debris around which further build up can accumulate. Accordingly, there is a need for devices, systems, and methods that continuously disinfect plumbing fixtures, preventing biofilm growth, and improving plumbing fixture performance.

### BRIEF DESCRIPTION OF THE DRAWINGS

Objects, features, and advantages of the present disclosure should become more apparent upon reading the following detailed description in conjunction with the drawing figures, in which:

FIGS. 1 and 2 illustrate perspective views of toilets in accordance with examples of the present disclosure. Specifically, FIG. 1 illustrates a toilet including a skirt and FIG. 2 illustrates a non-skirted toilet according to exemplary embodiments of the present disclosure.

FIG. 3 illustrates a biofilm prevention device in accordance with one example of the present disclosure.

FIG. 4 illustrates a biofilm prevention device in accordance with one example of the present disclosure.

FIG. 5 illustrates a biofilm prevention device in accordance with one example of the present disclosure.

FIG. 6 illustrates a biofilm prevention device coupled to a sink in accordance with one example of the present disclosure.

FIG. 7 illustrates a biofilm prevention device in accordance with one example of the present disclosure.

FIG. 8. Illustrates a biofilm prevention device disposed in a toilet tank in accordance with one example of the present disclosure.

FIG. 9 illustrates a biofilm prevention system in accordance with one example of the present disclosure.

FIG. 10 illustrates a flow chart for performing biofilm disinfection in accordance with one example of the present disclosure.

FIG. 11 illustrates an apparatus for facilitating biofilm disinfection in accordance with one example of the present disclosure.

The figures illustrate certain exemplary embodiments of the present disclosure in detail. It should be understood that the present disclosure is not limited to the details and methodology set forth in the detailed description or illustrated in the figures. It should be understood that the terminology used herein is for the purposes of description only and should not be regarded as limiting.

### DETAILED DESCRIPTION

The term “plumbing fixture” refers to an apparatus that is connected to a plumbing system of a house, building, or another structure. The term “plumbing fixture” may include toilets, bidets, faucets, sinks, showerheads, bathtubs, hot tubs, urinals, and dishwashers and their associated drain pipes and/or p-traps. The term “bathroom fixture” and “kitchen fixture” may more specifically refer to individual types of plumbing fixtures found in the bathroom or kitchen, respectively, and these terms may be overlapping in certain examples (e.g., faucets).

The biofilm prevention devices described herein may be configured to disinfect any of the above described plumbing fixtures, preventing biofilm growth. Specifically, the biofilm prevention devices described herein may be configured to provide or supply a chlorine dioxide solution to any of the above described plumbing fixtures, disinfecting the plumbing fixtures preventing biofilm growth (e.g., accumulation). In some examples, the biofilm prevention devices described herein may be integrally formed with one or more plumbing fixtures. For example, a biofilm prevention device may be integrally formed with the drain pipe or p-trap of the plumbing fixture. However, it is also envisioned that the biofilm prevention device can be sold as an after-market add-on product, capable of being installed on a plumbing fixture by a party other than the plumbing fixture's manufacturer.

Described herein are device, systems, and methods for disinfecting plumbing fixtures, preventing biofilm growth. Specifically, the biofilm prevention devices described herein may mix a dry chlorine dioxide precursor with water in-situ (e.g., at or in close proximity to the plumbing fixture) to create a chlorine dioxide solution. The chlorine dioxide solution may then be applied to the plumbing fixture, disinfecting the plumbing fixture, and preventing biofilm growth. The biofilm prevention device may be configured to create (e.g., mix, batch) the chlorine dioxide solution and provide it to the plumbing fixture at regular intervals so as to continuously prevent biofilm growth in the plumbing fixture.

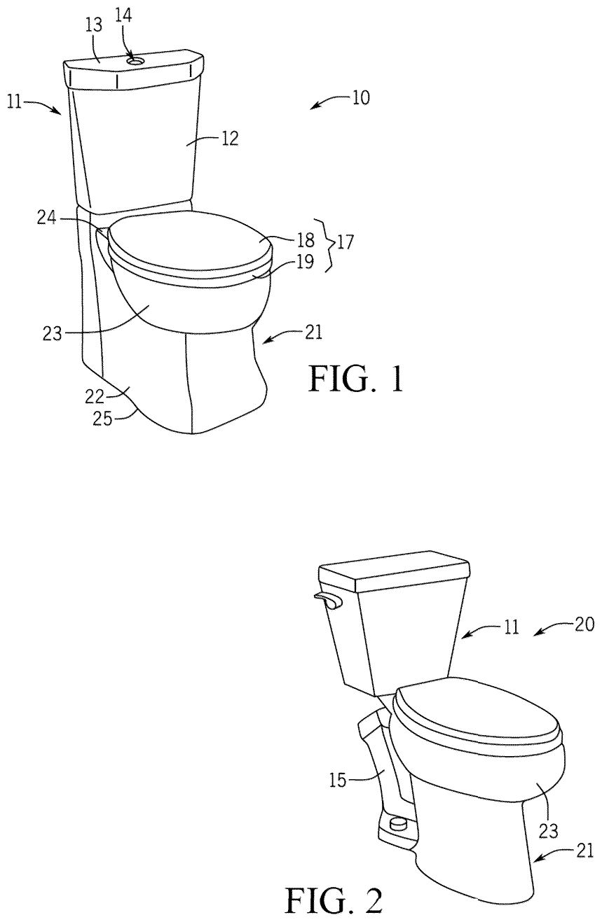

FIGS. 1 and 2 illustrate toilets according to examples of the present disclosure. FIG. 1 illustrates an exemplary embodiment of a skirted toilet 10 that includes a tank 11, a pedestal 21 (or base), and a seat assembly 17. The tank 11 may include a reservoir 12 for storing the water used during operational (or flushing) cycles, a lid (or cover) 13 for providing selective access into the reservoir 12, and an actuator 14 that is configured to initiate an operational cycle when activated. The actuator 14 or flush mechanism may be a button configured to activate when depressed (or pulled) a predetermined distance or when touched, a lever configured to activate when rotated a predetermined angular travel, or any suitable device configured to activate based upon an input manipulation by a user.

It should be noted that the shapes and configurations of the tank, pedestal, seat assembly, and the internal components (including the trapway and other features) may vary from the embodiments shown and described herein, and that the embodiments disclosed herein are not intended as limitations. It should be noted that various components of the toilet may be made of a vitreous material such as clay. It should be noted that various components of the toilet may be polymeric and/or over molded or otherwise fixed to the toilet. It should be noted, for example, that although the exemplary embodiment of the toilet 10 is shown configured with the tank 11 formed separately from the pedestal 21 and later coupled to the pedestal, the tank may be integrally formed with the pedestal as a one-piece design. In other words, the toilet may be a one-piece design, a two-piece design, or have any suitable configuration. The toilet disclosed herein may have a wide variety of skirted toilet configurations, and all such configurations are intended to be encompassed herein. The following description of various toilet features is therefore intended as illustration only of one possible embodiment, and it should be understood by those reviewing the present description that similar concepts or features may be included in various other embodiments.

The tank 11 may include an inlet opening configured to receive water from a coupled water supply, such as from a hose (e.g., line, tube). The tank 11 may also include an inlet valve assembly or other device configured to control the flow of water from the water supply into the tank through the inlet opening. Within the tank 11 may be provided a float device for controlling the inlet valve assembly, such as by opening the valve to refill the reservoir 12 of the tank 11 after an operational cycle and closing the valve when the water in the reservoir 12 reaches a preset volume or height. The tank 11 may also include an outlet opening configured to transfer (e.g., conduct) the water stored in the reservoir 12 of the tank to the pedestal 21 upon activation of the actuator 14. The pedestal 21 may include toilet bowl 23. The tank 11 may include an outlet valve assembly or other device configured to control the flow of water from the reservoir 12 into the pedestal 21 through the outlet opening.

The pedestal 21 (or base) of the toilet 10 may include a wall 22 having any suitable shape that is configured to form a bowl 23 having an opening formed by an upper rim at the top of the opening. The pedestal 21 may also be configured to include a plurality of walls having varying shapes that together form a bowl having an opening formed by a rim. The wall 22 of the pedestal may extend downward and/or rearward from the bowl 23 to form a lower portion 25 configured to support the pedestal 21 and the toilet 10. The lower portion 25 may be formed by the end (e.g., lower rim) of the wall 22, or may include a member that extends generally in a horizontal plane from one or more than one end of the wall. The pedestal 21 may also include a top member 24 that extends between two sides of the wall 22 (or between two opposing walls) and is provided rearward (or behind) the bowl 23, wherein the top member 24 forms a plateau for supporting the tank 11, such as the bottom surface of the reservoir 12 of the tank 11. The top member 24 may include an inlet opening that may be aligned with the outlet opening of the tank 11, such as when the tank 11 is coupled to (or resting above) the pedestal 21, wherein water is selectively transferred (e.g., conducted) from the tank 11 through the outlet opening of the tank to the pedestal 21 through the inlet opening of the pedestal 21, when the toilet is activated through the actuator 14. The outlet valve assembly may control the flow of water from the tank to the pedestal. The toilet may also include a gasket or seal that is provided between the tank 11 and the pedestal 21 to prohibit leaking. For example, a gasket may be provided between the outlet opening of the tank and the inlet opening of the pedestal to prohibit leaking between the tank and the pedestal.

The plateau or upper surface formed by the top member 24 of the pedestal 21 may also provide for coupling of the seat assembly 17 to the pedestal 21 of the toilet 10. For example, the top member 24 may include one or more than one opening, wherein each opening is configured to receive a fastening device (e.g., bolt, screw, etc.) to couple (e.g., attach) the seat assembly 17 to the top member 24 of the pedestal 21. As another example, the top member 24 may include one or more than one fastening device (e.g., bolts, recessed nuts, etc.) integrally formed therein (i.e., already provided connected or coupled to the pedestal 21), wherein the fastening device may be used to couple or secure at least a portion of the seat assembly 17 to the pedestal 21. The seat assembly 17 may include a hinge, hinge shoulders configured to receive a fastener, a seat coupled to the hinge and a cover coupled to the hinge.

The bowl 23 of the pedestal 21 may be configured to include a receptacle (e.g., sump) and an outlet opening, wherein the water and waste is collected in the receptacle until being removed through the outlet opening, such as upon activation of the actuator 14. The pedestal 21 may also include a pedestal internal passageway, such as a trapway, that connects the outlet opening or discharge outlet of the bowl 23 to a drain or soil pipe. The passageway, or trapway, generally includes a first portion, a second portion, and a weir separating the first and second portions. The first portion of the passageway may extend from the outlet opening of the bowl 23 at an upwardly oblique angle to the weir. The second portion of the passageway may extend from the weir downwardly to the exiting device, such as the drain or soil pipe.

Between operational cycles (e.g., flush cycles) of the toilet 10, the water (and waste) is collected in the first portion of the trapway (in addition to the receptacle of the bowl), such that the weir prohibits the water from passing past the weir and into the second portion of the trapway. A flushing cycle may begin upon activation of the actuator 14. Upon activation of the actuator, additional water may be discharged into the bowl 23 of the pedestal 21, resulting in the flushing action and waste removal through the soil pipe. For example, water may be discharged into the bowl from one or more rim outlets located in or below a rim of the toilet and/or a sump jet disposed in a sump (e.g., first part of the trapway) of the toilet. The rim outlets and/or the sump jet may include one or more of the fluidic devices described herein. The flushing cycle may include generation of a siphon to assist the flushing action and waste removal.

The seat assembly 17 may include a cover member 18 (e.g., lid), a seat member 19 (e.g., ring member), and a hinge. The seat member 19 may be configured to include an annular member that encircles an opening, wherein the annular member provides a seating surface for the user of the toilet 10. The seat member 19 may also be pivotally coupled (e.g., attached) to the hinge, wherein the seat member may rotate (or pivot) about the hinge, such as between a first lowered or seated position and a second raised or upright position. The cover member 18 may be configured to be round, oval, or any other suitable shape. Typically, the profile or shape of the outer surface of the cover member will be configured to match (i.e., to be substantially similar) to the profile of the outer surface of the seat member to improve the aesthetics of the seat assembly and toilet. The cover member 18 may also be coupled to the hinge, wherein the cover member may rotate (or pivot) about the hinge, such as between a first lowered or down position and a second raised or upright position. The cover member 18 may be provided above the seat member in the down position to thereby cover the opening of the seat member 19, as well as to conceal the inside of the bowl 23 of the pedestal 21. The cover member 18 may be configured to rest against the outside surface of the tank 11, when the cover member 18 is in the upright position, such that the cover member 18 remains in the upright position in order for a user to sit upon the seat member 19.

FIG. 2 illustrates a non-skirted toilet 20 according to another exemplary embodiment of the present disclosure. The internal components, including the trapway 15, are visible in the pedestal 21 of non-skirted toilet 20. It should be noted that the devices, methods, and systems described herein may include and/or be used with both skirted and non-skirted toilets. It should further be noted that devices, methods, and systems described herein may include or be used with both toilets including tanks and tankless toilets. A waterline may supply a tankless toilet with water during a flush cycle.

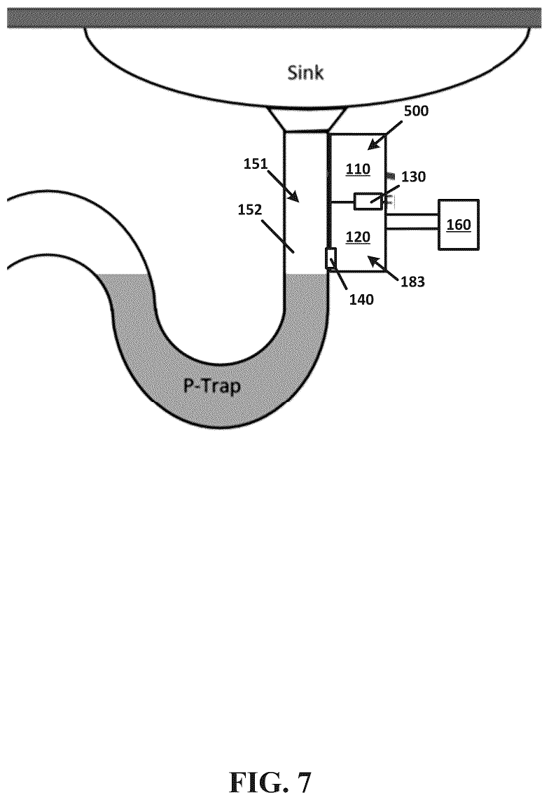

Referring generally to FIGS. 1 and 2, the toilet 10, 20 may be coupled to or include a biofilm prevention device as described herein. For example, as illustrated in FIG. 7 and described hereinbelow, the tank 11 may include a biofilm prevention device configured to disinfect the reservoir 12, bowl 23, sump, trapway and/or drain pipe, preventing biofilm growth.

According to other examples, a biofilm prevention device as described herein may provide the chlorine dioxide solution to a flow of water provided to the bowl of an in-line (i.e., tankless) toilet during an operational cycle of the toilet, disinfecting a bowl 23, sump, trapway, drainpipe, and the like of the toilet. An operational cycle may be performed absent use of the toilet in order to provide the chlorine dioxide solution to the toilet. For example, an operational cycle for application of the chlorine dioxide solution may be automatically performed at during low use hours (e.g., at night).

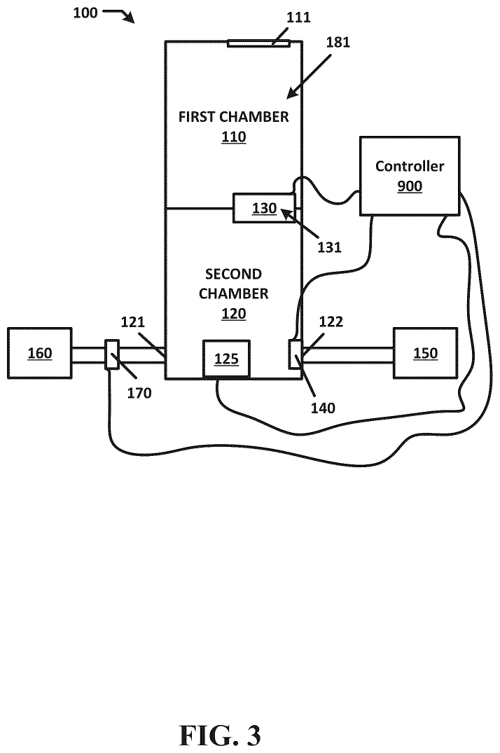

Referring to FIGS. 3-5, biofilm prevention devices 100, 200, 300 in accordance with multiple examples of the present disclosure are illustrated. Each of the biofilm prevention devices 100, 200, 300 includes a first chamber 110, a second chamber 120, a conveyor 131 (e.g., first valve 130) disposed between the first chamber 110 and the second chamber 120, and a second valve 140 disposed at an outlet 122 of the second chamber 120. In some examples, the biofilm prevention devices 100, 200, 300 may include a housing 181, 182 including one or more of the first chamber 110, second chamber 120, first valve 130, and second valve 140. The housing 181, 182 may include one or more walls or panels defining a size, shape, and the like of the first chamber 110 and/or the second chamber 120. The housing 181, 182 may further include one or more walls of panels defining an exterior surface of the housing 181, 182 and/or the biofilm prevention device 100, 200, 300. The biofilm prevention devices 100, 200, 300 may be configured to provide or supply a chlorine dioxide solution to a plumbing fixture 150.

The first chamber 110 is configured to store a dry precursor. The dry precursor may generate or produce chlorine dioxide (ClO₂) when mixed with water (H₂O). The dry precursor may include an acid source (e.g., sodium acid sulfate), a chlorine source (e.g., sodium chlorite, sodium chloride), and a stabilizing compound (e.g., magnesium sulfate). In some examples, the dry precursor may comprise a powder. In other examples, the dry precursor may comprise one of more tablets or pellets. In some examples, each tablet may include a known or predetermined quantity of dry precursor. A controlled or predetermined quantity (e.g., one, two, three) of tablets or a predetermined quantity (e.g., volume, mass) of dry precursor in powder form may be mixed with a controlled or predetermined (e.g., controlled) volume of water. The predetermined (e.g., controlled) quantity of dry precursor (in powder or tablet form) may be mixed with a predetermined (e.g., controlled) volume of water to create an aqueous solution including gaseous chlorine dioxide having a desired concentration of chlorine dioxide. The first chamber 110 may be water proof. Accordingly, water may be prevented from entering the first chamber 110 and reacting with the dry precursor.

In some examples, as illustrated in FIG. 1, the first chamber 110 may be disposed in a housing 181 of the biofilm prevention device 100. As illustrated in FIG. 1, the housing 181 includes the first chamber 110, second chamber 120, first valve 130, and second valve 140. In these examples, the housing 181 or first chamber 110 may include a gate or door 111 configured to selectively provide access to the first chamber 110 (e.g., to refill the first chamber 110 with dry precursor). When in the closed position, the door 111 may engage the housing 181 or the first chamber 110 forming a watertight seal that prevents water from entering the first chamber 110.

In other examples, as illustrated in FIG. 2, the first chamber 110 is disposed in a cartridge 190. The cartridge 190 that is removably coupled to the housing 182. In these examples, the housing 182 may include the second chamber 120, first valve 130, and second valve 140. In these examples, the cartridge 190 may be replaced when all of the dry precursor has been used.

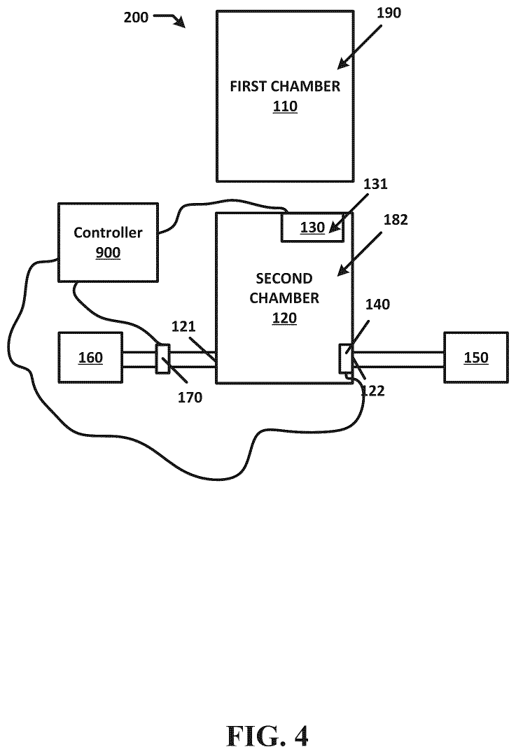

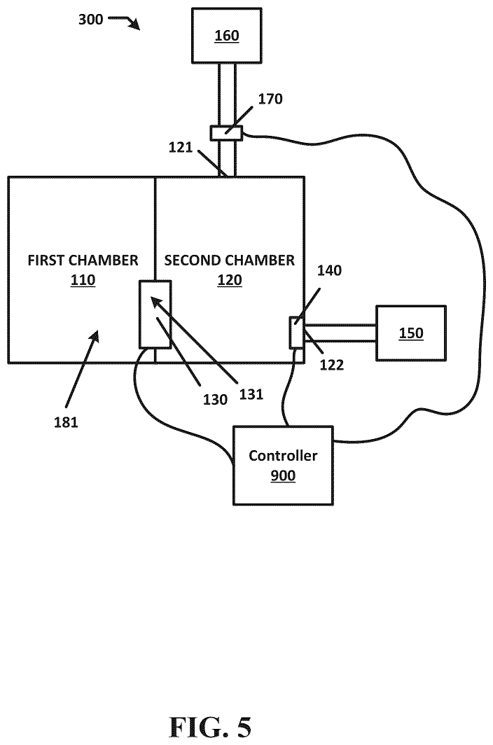

Referring generally to FIGS. 3-5, the mixing or second chamber 120 may be disposed in the housing 181, 182. The second chamber 120 may include an inlet 121 and/or an outlet 122. The second chamber 120 may be configured to receive a volume of water (e.g., through the inlet 121). In some examples, the second chamber 120 may be configured to receive a predetermined (e.g., controlled, metered) volume of water configured to create a chlorine dioxide solution having a desired (e.g., controlled) volume and/or concentration. Additionally, the second chamber 120 may be configured to receive dry precursor (e.g., through the first valve 130). In some examples, the second chamber 120 may be configured to receive a predetermined quantity of dry precursor configured to create a chlorine dioxide solution having a desired volume and/or concentration. The second chamber 120 may be configured to contain the volume of water and dry precursor while they react, creating the chlorine dioxide solution. In some examples, as illustrated in FIGS. 3 and 4, the first chamber 110 may be disposed above the second chamber 120. Storing the dry precursor (e.g., in the first chamber 110) above the second chamber 120 may prevent water from entering the first chamber 110, for example through first valve 130. In other examples, as illustrated in FIG. 5, the first chamber 110 may be disposed adjacent to the second chamber 120. In some examples, the second chamber 120 may further include a mixing or agitating device 125 configured to mix the dry precursor and water. The agitating device 125 may be configured to stir or vibrate the water and dry precursor, facilitating the chemical reaction between water and dry precursor.

Referring generally to FIGS. 3-5, the conveyor 131 (e.g., first valve 130) is disposed between the first chamber 110 and the second chamber 120. The conveyor 131 (e.g., first valve 130) may be configured to provide a predetermined quantity of dry precursor to the second chamber 120. In some examples, the conveyor 131 may be a first valve 130. The first valve 130 may a positive displacement pump such as screw pump, gear pump, vane pump, or the like. In other examples, the conveyor 131 may comprise a feeding mechanism including one or more elastic members (e.g., springs) configured to provide a predetermined quantity of dry precursor to the second chamber 120. For example, the conveyor 131 may comprise a feeding mechanism including a spring configured to provide one or more tablets or pellets of dry precursor to the second chamber 120. The conveyor 131 may be connected to a controller 900 and may be configured to provide a predetermined quantity of dry precursor to the second chamber 120 in response to control signals and/or electric current received from the controller 900. In some examples, the controller 900 may be disposed in the housing 181, 182. The first valve 130 may be disposed in the housing 181, 182 and/or the second chamber 120 of the biofilm prevention device 100, 200, 300.

Referring generally to FIGS. 3-5, the second valve 140 may be disposed at the outlet 122 of the second chamber 120. The second valve 140 may be configured to control a flow of chlorine dioxide solution from the second chamber 120 to the plumbing fixture 150. In some examples, the second valve 140 may be disposed in the second chamber 120 and/or the housing 181, 182. The second valve 140 may be connected to a controller 900 and may be configured to selectively provide (e.g., open/close) the chlorine dioxide solution to the plumbing fixture 150 in response to control signals and/or electric current received from the controller 900.

Referring generally to FIGS. 3-5, in some examples, the biofilm prevention device may include a third valve 170 configured to control a flow of water from a water supply 160 to the second chamber 120. The third valve 170 may be configured to control a quantity or volume of water flowing therethrough, and thus, a quantity or volume of water supplied to the second chamber 120. The third valve 170 may be connected to a controller 900 and may be configured to provide a predetermined volume of water to the second chamber 120 in response to control signals and/or electric current received from the controller 900. For example, the controller 900 may provide one or more control signals and/or electric current to the third valve 170, such that the third valve remains open for a predetermined period of time corresponding to the desired volume of water. In some examples, the third valve 170 may be a pump, for example, a positive displacement pump configured to provide (e.g., pump) the predetermined volume of water to the second chamber. In some examples, the water supply 160 may be a building (e.g., residential, commercial) plumbing network. In other examples, water supply 160 may be a well or another source.

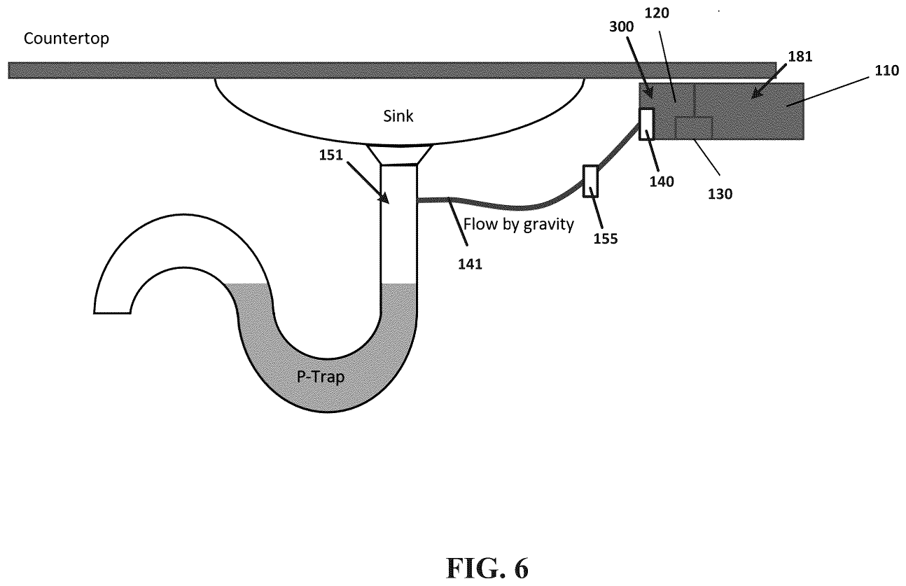

Referring to FIG. 6, in some examples, the biofilm prevention device 300 may be coupled to a sink 151. In some examples, as illustrated in FIG. 6, the biofilm prevention device 300 may be coupled to a countertop, vanity, or cabinet proximate to the sink 151. In some examples, the sink may be disposed in the bathroom. In other examples, the sink 151 may be disposed in a kitchen. In some examples, as illustrated in FIG. 6, the biofilm prevention device 300 may supply or provide a chlorine dioxide solution directly to a P-trap of the sink 151. In other examples, the biofilm prevention device 300 may provide a chlorine dioxide solution directly to the basin of the sink 151. In some examples, as illustrated in FIG. 6, a hose 141 may fluidly couple the second valve 140 and the P-trap of the sink 151. In other examples the hose 141 may fluidly couple the second valve 140 and the basin of the sink 151. In some examples, gravitational potential energy may be used to convey the chlorine dioxide solution from the second chamber 120 through the second valve 140 to the sink 151. In other examples, a pump 155 may be disposed between the second valve 140 and the sink 151.

Referring to FIG. 7, in some examples, the biofilm prevention device 500 may be integrally formed with the plumbing fixture (e.g., 150). For example, as illustrated in FIG. 7, the plumbing fixture may be a sink 151 and the biofilm prevention device 500 may be integrally formed with a P-trap of the sink 151. Specifically, as illustrated in FIG. 7, the biofilm prevention device 500 may be integrally formed with a conduit 152 of the P-trap of the sink 151. As illustrated in FIG. 7, the conduit 152 is disposed in-line with the sink 151.

In some examples, the biofilm prevention device 500 may include a first chamber 110 that is included in the housing 183, as described above with respect to the biofilm prevention device 100. In other examples, biofilm prevention device 500 may include a first chamber 110 disposed in a cartridge (e.g., 190) that is removably coupled to the housing 183, as described above with respect to the biofilm prevention device 200.

As illustrated in FIG. 7, the housing 183 may be integrally formed with or attached to the conduit 152. For example, the housing 183 and conduit 152 may be integrally formed using injection molding, compression molding, resin transfer molding, gravity casting, or the like. As illustrated in FIG. 7, the second valve 140 may be disposed between the second chamber 120 and the conduit 152. Accordingly, the biofilm prevention device 500 may provide a chlorine dioxide solution to from the second chamber 120, through the second valve 140 and directly into the conduit 152.

The biofilm prevention device 500 may be sold as an after-market add-on product, capable of being installed on the sink 151 by a party other than the sink's manufacturer. Specifically, a pipe or conduit in the P-trap of the sink may be replaced by the biofilm prevention device. Similarly, the biofilm prevention device 500 may be installed on a different plumbing fixture, such as, a drain pipe or P-trap for a shower, a bath tub, hot tub, a bidet, a urinal, or a dishwasher.

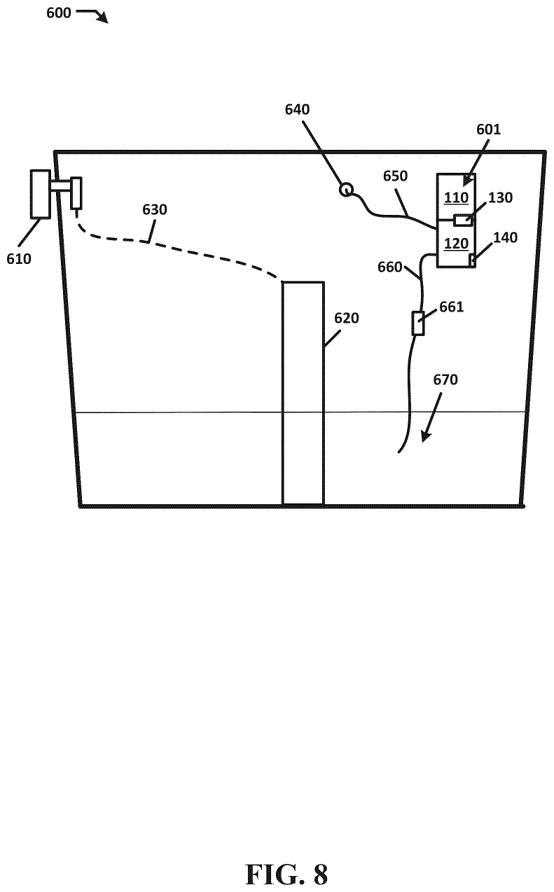

Referring to FIG. 8, a toilet tank 600 including a biofilm prevention device 601 in accordance with one example of the present disclosure is illustrated. As illustrated in FIG. 8, the biofilm prevention device 601 may be disposed in the toilet tank 600. The biofilm prevention device 601 may be substantially similar to the biofilm prevention devices 100, 200, 300 described above with respect to FIGS. 3-5. Specifically, the first chamber 110 may be disposed within the housing (e.g., 181) or may be disposed within a cartridge (e.g., 190 removably coupled to the housing (e.g., 182). Additionally, the first chamber 110 may be disposed on top of or next to the second chamber 120.

Referring generally to FIG. 8, the toilet tank 600 includes a mechanical actuator 610 coupled to a flush valve 620 via a chain 630 configured to initiate an operational (e.g., flush) cycle of a toilet. However, the present disclosure is not limited thereto, and the tank 600 may instead include a push button, capacitive sensor, or the like configured to send a control signal and/or electric current to a flush valve to initiate an operational cycle of the toilet. An electronic actuator may be connected to controller 900 to initiate an operational cycle of the toilet. Additionally, the tank 600 includes an inlet opening and/or inlet valve 640 configured to receive a flow of water refilling the tank 600 after an operational cycle of the toilet.

In some examples, the second chamber 120 of the biofilm prevention device 601 may be configured to receive a predetermined volume of water through a tube 650 fluidly coupling the inlet opening and/or inlet valve 640 and the second chamber 120. In some examples, an inlet valve 640 may control a volume of water supplied to the second chamber 120. In other examples, another valve (e.g., third valve 170) may be disposed between the inlet opening and/or inlet valve 640 and the second chamber 120 and may control a volume of water supplied to the second chamber 120. In other examples, the biofilm prevention device may include a hose 660 extending (e.g., downward) into a volume of water 670 stored in the tank and a pump 661 configured to supply (i.e., pump) a predetermined volume of water into the second chamber 120.

Referring to FIG. 8, after the chlorine dioxide solution is mixed or created in the second chamber 120, the second valve 140 may open allowing the chlorine dioxide solution to flow (e.g., under the assistance of gravity) into the tank 600 of the toilet. The chlorine dioxide solution may disinfect the tank 600 of the bowl, while the volume of water 670 and chlorine dioxide solution are disposed in the tank 600. During an operational cycle of the toilet, the chlorine dioxide solution may disinfect one or more of a rim channel, a rim outlet, a bowl, a sump, a trapway, and a drain pipe as the chlorine dioxide solution flows through the toilet. In some examples, chlorine dioxide solution may be supplied to the tank (e.g., under direction of the controller 900) and an operational cycle of the toilet may be initiated (e.g., under direction of the controller 900) when the toilet has not been used by a user (e.g., at night or during low use periods) for purposes of disinfecting the toilet and preventing biofilm growth.

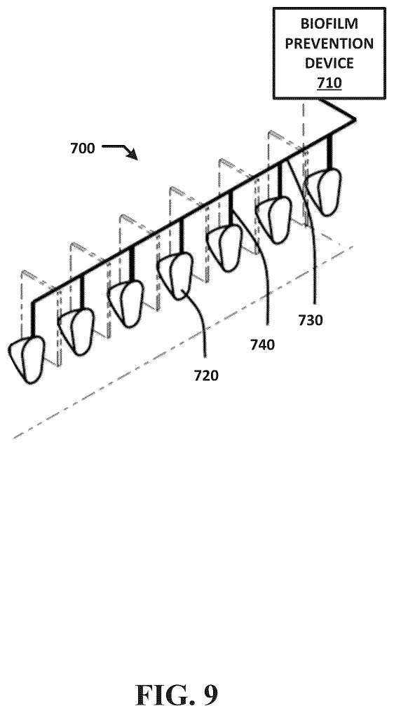

Referring to FIG. 9, a biofilm prevention system 700 for disinfecting and preventing biofilm growth in a plurality of plumbing fixtures is illustrated. As illustrated in FIG. 9, in some examples, a single biofilm prevention device 710 may be configured to provide a chlorine dioxide solution to two or more plumbing fixtures. For example, referring to FIG. 9, the biofilm prevention device 710 may be configured to provide a chlorine dioxide solution to a plurality of urinals 720. The biofilm prevention device 710 may be disposed, for example, in a wall, cabinet, closet, or the like proximate to the plurality of urinals 720.

The biofilm prevention system 700 may include a main supply line 730 and a plurality (i.e., two or more) of secondary supply lines 740. The main supply line 730 may be in fluid communication with the second chamber 120 of the biofilm prevention device 710 via the second valve 140. The main supply line 730 may receive the chlorine dioxide solution from the second chamber 120 through the second valve 140 when the second valve 140 is in an open position. Each of the secondary supply lines 740 may extend between and be in communication with the main supply line 730 and a urinal 720. The secondary supply lines 740 may convey the chlorine dioxide solution from the main supply line 730 to the urinals 720. In some examples, the main supply line 730 and secondary supply lines 740 may be configured to supply a flow of water to the plurality of urinals, for example, during or between operational cycles of the urinals 720.

As illustrated in FIG. 9, the biofilm prevention device 710 may be configured to provide a chlorine dioxide solution to all of the urinals 720 via the main supply line 730 and secondary supply lines 740. Similarly, a single biofilm prevention device 710 may be in fluid communication with and configured to provide a chlorine dioxide solution to a plurality of different plumbing fixtures, such as, a plurality of sinks, toilets, showers, or the like.

In some examples, a single biofilm prevention device may be connected to two or more different plumbing fixtures. For example, a single biofilm prevention device may be in communication with two or more of a sink, a shower, a toilet, a dishwasher, and the like, disposed in a commercial or residential bathroom or kitchen. A single biofilm prevention device may be configured to provide a chlorine dioxide solution to each of the plumbing fixtures, disinfecting the plumbing fixtures and preventing biofilm growth.

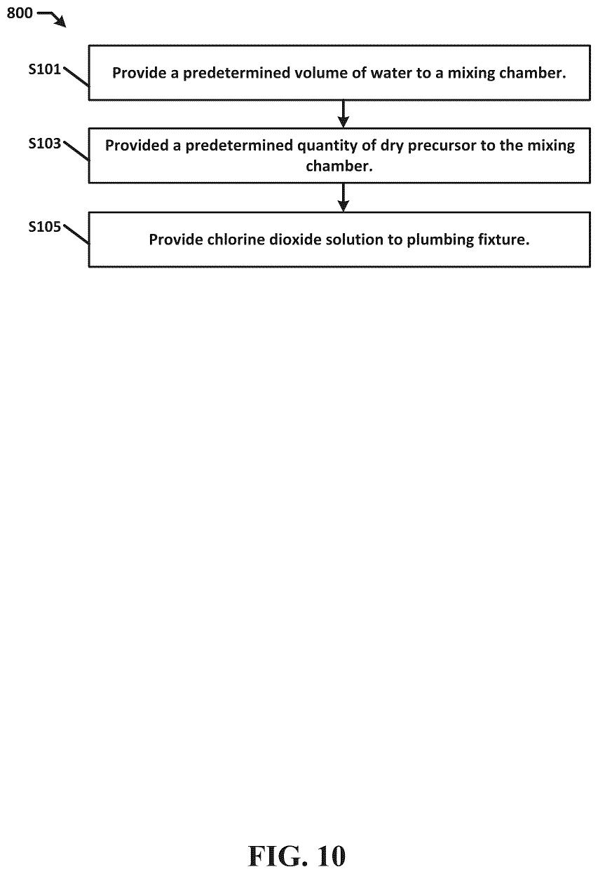

Referring to FIG. 10, a flow chart 800 for disinfecting a plumbing fixture and preventing biofilm growth in accordance with one example of the present disclosure is illustrated. The flow chart 800 may be used with any of the biofilm prevention devices 100, 200, 300, 500, 601, 710 described herein. Additional, different, or fewer acts may be provided.

In a first act S101, a predetermined (e.g., controlled, metered) volume of water is provided to a mixing chamber (i.e., second chamber 120) of the biofilm prevention device. The predetermined volume of water may be volume of water required to create a chlorine dioxide solution having a desired volume and concentration when mixed with a predetermined (e.g., controlled, metered) quantity of dry precursor.

In some examples, the flow chart 800 may additionally include the act of opening a water supply valve (i.e., second valve 140), wherein the predetermined volume of water is provided to mixing chamber through the water supply valve (and/or subsequently closing the water supply valve). The water supply valve may be opened for a period of time corresponding to or required for the predetermined volume of water to flow into the mixing chamber. In some examples, the water supply valve may be connected to a controller 900. In these examples, the water supply valve may open and/or close in response to a control signal and/or electric current received from the controller.

In a second act S103, a predetermined (e.g., controlled, metered) quantity of dry precursor is provided to the mixing chamber of the biofilm prevention device. The dry precursor may be provided to the mixing chamber from a storage chamber (i.e., first chamber 110). The predetermined quantity of dry precursor may be a quantity of dry precursor required for create a chlorine dioxide solution having a desired volume and concentration when mixed with the predetermined volume of water.

In some examples, when the predetermined quantity of dry precursor is provided to the mixing chamber, the predetermined volume of water may already be in the mixing chamber. Accordingly, when the dry precursor is provided to the mixing chamber, the dry precursor and the water may react with one another creating a chlorine dioxide solution having a desired (e.g., controlled) volume and concentration of chlorine dioxide. In some examples, the flow chart 800 may additionally include actuating a mixing or agitating device configured to mix the dry precursor and the water. In some examples, the predetermined quantity of dry precursor may be a volume or mass of dry precursor. In other examples, the predetermined quantity of dry precursor may be a number of tablets (e.g., one, two, three) of dry precursor.

In some examples, the flow chart 800 may additionally include the act of opening a precursor valve (i.e., 120), wherein the predetermined quantity of dry precursor is provided to the mixing chamber through the precursor valve. In some examples, the precursor valve may be a positive displacement valve, such as, a screw pump and may perform one or more operation cycles, providing the predetermined quantity of dry precursor to the mixing chamber. Each operational cycle of the precursor valve may provide the same (known) volume, mass, or quantity of tablets of dry precursor. In some examples, the precursor valve may be connected to a controller 900. In these examples, the precursor valve may actuate in response to a control signal and/or electric current received from the controller 900.

In a third act S105, the chlorine dioxide solution is provided to a plumbing fixture (e.g., 150). The chlorine dioxide solution may be provided to various different parts of a plumbing fixture. For example, in the case of a sink, the chlorine dioxide solution may be supplied to a basin of the sink and/or to a P-trap or drain pipe of the sink. In some examples, the chlorine dioxide solution may be provided to the plumbing fixture through an outlet valve (i.e., second valve) and on or more hoses connecting the mixing chamber, second valve, and/or plumbing fixture. In other examples, the chlorine dioxide solution may be provided through the outlet valve, directly into the plumbing fixture.

In some examples, the flow chart 800 may additionally include the act of opening the outlet valve (i.e., 140), wherein the chlorine dioxide solution is provided to the plumbing fixture through the outlet valve. In some examples, the outlet valve may be connected to a controller 900. In these examples, the outlet valve may actuate (e.g., open/close) in response to a control signal and/or electric current received from the controller 900.

In some examples, the flow chart 800 may further include coupling a cartridge to the mixing chamber. The cartridge may include the dry precursor (e.g., tablets or powder) required to create the chlorine dioxide solution. The flow chart 800 may include coupling a cartridge to the mixing chamber when the first chamber is disposed in a cartridge that is removably coupled to the housing or second chamber. In these examples, the cartridge may be a disposable cartridge including the dry precursor.

In some examples, the flow chart 800 may be used to disinfect a plumbing fixture and prevent biofilm growth at a time of the day in which it is not likely that a user is using or proximate to the plumbing fixture. For example, the flow chart 800 may be used at night to disinfect a plumbing fixture when potential users are likely asleep. In some examples, controller 900 may be programed to initiate the flow chart 800 at night or during other low-usage time periods.

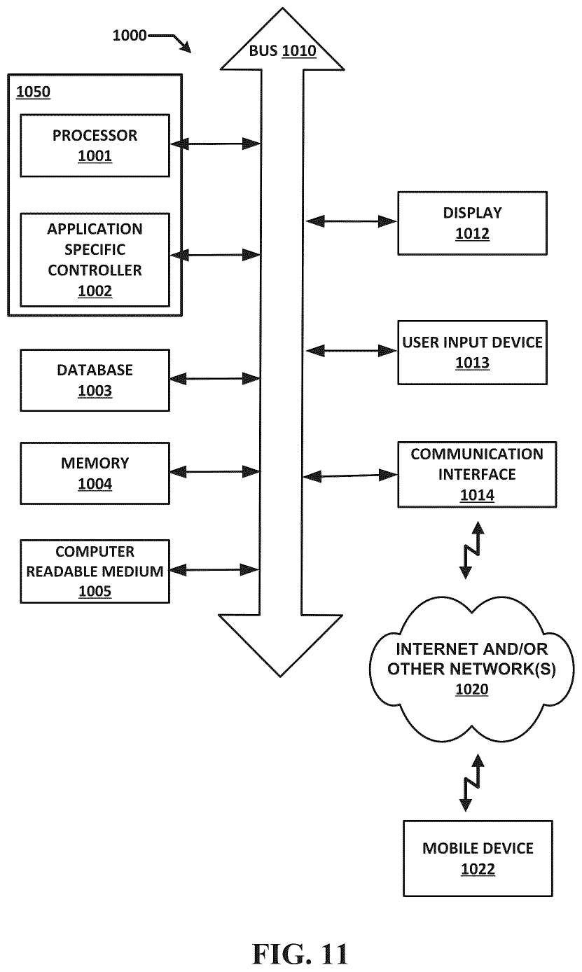

Referring to FIG. 11, an apparatus 1000 for facilitating an operational cycle (e.g., flow chart 800) of a biofilm prevention device 100, 200, 300, 601, 710 in accordance with one example of the present disclosure is illustrated. The apparatus 1000 includes a bus 1010 facilitating communication between a controller 1050 that may be implemented by a processor 1001 and/or application specific controller 1002 and one or more components including a database 1003, a memory 1004, a computer readable medium 1005, display 1012, a user input device 1013, and a communication interface 1014.

The contents of the database 1003 may include one or more predetermined volumes of water and one or more predetermined quantities of dry precursor required to create a chlorine dioxide solution of known volume and concentration. In some examples, the database 1003 may include one or more formulas or equations, for calculating a quantity of dry precursor and/or water required to create a chlorine dioxide solution of a selected concentration and/or volume. In some examples, the one or more formulas may be used to dynamically change a volume and/or concentration of chlorine dioxide solution supplied to the plumbing fixture. Additionally, the database may include one or more flow rates (e.g., through a valve 130, 140, 170), the area of one or more valves (e.g., 140, 170), and/or one or more time periods required for a known volume of fluid to travel through a valve (e.g., 140, 170). In some examples, the database 1003 may store an equation or equations for calculating a volume, flow rate, or time period relating to fluid provided or conveyed through the second valve 140 and third valve 170. In some examples, the database 1003 may store an equation or equations for calculating a concentration and/or volume of a chlorine dioxide solution.

The memory 1004 may be a volatile memory or a non-volatile memory. The memory 1004 may include one or more read only memory (ROM), random access memory (RAM), a flash memory, an electronic erasable program read only memory (EEPROM), or other type of memory. The memory 1004 may be removable from the apparatus 1000, such as a secure digital (SD) memory card.

The memory 1004 and/or the computer readable medium 1005 may include a set of instructions that can be executed to cause the controller to perform any one or more of the flow charts (e.g., 800) or computer-based functions disclosed herein. For example, the controller 1050 may send one or more controller signals and/or electric current to the first valve 130, second valve 140, and third valve 170 opening and/or closing the valves (i.e., such that a biofilm prevention device performs the flow chart 800). In some examples, the memory 1004 and/or the computer readable medium 1005 may include one or more sets of instructions. The one or more sets of instructions may correspond to, for example, one or more different volumes and/or concentrations of chlorine dioxide solution created and/or supplied to the plumbing fixture. The memory 1004 may store the one or more formulas or equations for determined a quantity of dry precursor and/or water for creation of a chlorine dioxide solution having a specific concentration and/or volume.

A user may initiate an operational cycle of the biofilm prevention device, input one or more volumes, and/or input one or more time periods relevant to an operational cycle using the display 1012 and/or user input device 1013. In some examples, a user may select a volume and/or concentration of chlorine dioxide solution using the display 1012 and/or user input device 1013. The display 1012 may comprise a screen and the user input device 1013 may comprise one or more buttons on the apparatus 1000. In some embodiments, the display 1012 and user input device 1013 may comprise a touch sensitive surface (i.e., a touch screen).

The communication interface 1014 may be connected to the network 1020, which may be the internet. In some embodiments, the network 1020 may be connected to one or more mobile devices 1022. The one or more mobile devices may be configured to send a signal to the communication interface 1014 via the network 1020. For example, a more mobile devices may send a signal to the communication interface to initiate an operational cycle of the biofilm prevention device or change one or more volumes of water, quantities of dry precursor, and/or concentrations of chlorine dioxide solution associated with an operation cycle of the biofilm prevention device.

The communication interface 1014 may include any operable connection. An operable connection may be one in which signals, physical connections and/or logical communications may be sent and/or received. An operable connection may include a physical interface, an electrical interface, and/or a data interface. The communication interface 1014 provides for wireless and/or wired communications in any known or later developed format.

When a component, device, element, or the like of the present disclosure is described as having a purpose or performing an operation, function, or the like, the component, device, or element should be considered herein as being “configured to” meet that purpose or to perform that operation or function.

As utilized herein, the terms “approximately,” “about,” “substantially”, and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the disclosure as recited in the appended claims.

It should be noted that the term “exemplary” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

The term “or,” as used herein, is used in its inclusive sense (and not in its exclusive sense) so that when used to connect a list of elements, the term “or” means one, some, or all of the elements in the list. Conjunctive language such as the phrase “at least one of X, Y, and Z,” unless specifically stated otherwise, is understood to convey that an element may be either X, Y, Z; X and Y; X and Z; Y and Z; or X, Y, and Z (i.e., any combination of X, Y, and Z). Thus, such conjunctive language is not generally intended to imply that certain embodiments require at least one of X, at least one of Y, and at least one of Z to each be present, unless otherwise indicated.

References herein to the positions of elements (e.g., “top,” “bottom,” “above,” “below”) are merely used to describe the orientation of various elements in the FIGURES. It should be noted that the orientation of various elements may differ according to other exemplary embodiments, and that such variations are intended to be encompassed by the present disclosure.

Although the figures and description may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

It is important to note that the construction and arrangement of the system as shown in the various exemplary embodiments is illustrative only. Additionally, any element disclosed in one embodiment may be incorporated or utilized with any other embodiment disclosed herein. Although only one example of an element from one embodiment that can be incorporated or utilized in another embodiment has been described above, it should be appreciated that other elements of the various embodiments may be incorporated or utilized with any of the other embodiments disclosed herein.

What is claimed is:

1. A biofilm prevention device for a plumbing fixture, the device comprising:
a first chamber configured to store a dry precursor;
a second chamber configured to receive a predetermined volume of water;
a conveyor disposed between the first chamber and the second chamber, the conveyor configured to convey a predetermined quantity of dry precursor into the second chamber; and
an outlet valve disposed at an outlet of the second chamber, the outlet valve configured to control a flow of chlorine dioxide solution to the plumbing fixture coupled to a sink drain pipe, a shower drain pipe, a urinal drain pipe, or a bathtub drain pipe.

2. The device of claim 1, wherein the flow of chlorine dioxide solution is provided upstream of a P-trap coupled to the sink drain pipe, the shower drain pipe, the urinal drain pipe, or the bathtub drain pipe.

3. The device of claim 1, further comprising:
a water supply valve disposed between a water supply and the second chamber, the water supply valve configured to provide the predetermined volume of water to the second chamber.

4. The device of claim 1, wherein the first chamber comprises a cartridge removably coupled to the second chamber.

5. The device of claim 4, wherein the dry precursor comprises a plurality of tablets.

6. The device of claim 5, wherein the flow of chlorine dioxide solution is configured to disinfect the plumbing fixture.

7. The device of claim 1, wherein the flow of chlorine dioxide solution to the plumbing fixture controlled by the outlet valve flows to two or more plumbing fixtures.

8. The device of claim 1, wherein the first chamber is disposed above the second chamber.

9. The device of claim 1, wherein the outlet valve includes a screw pump.

10. A biofilm prevention device, the device comprising:
a first chamber configured to store a dry precursor;
a second chamber configured to receive a predetermined volume of water;
a first valve disposed between the first chamber and the second chamber, the first valve configured to convey a predetermined quantity of dry precursor into the second chamber;
a conduit coupled to the second chamber, the conduit being in-line with one or more plumbing fixtures; and
a second valve disposed at an outlet of the second chamber, the second valve configured to control a flow of chlorine dioxide solution to the conduit.

11. The device of claim 10, wherein the conduit is a p-trap for one of a sink, a shower, a toilet, and a urinal.

12. The device of claim 10, further comprising a third valve coupled to the second chamber, the third valve configured to provide the predetermined volume of water to the second chamber.

13. The device of claim 10, wherein the first chamber is disposed above the second chamber.

14. The device of claim 13, wherein the first chamber comprises a cartridge removably coupled to the second chamber.

15. The device of claim 10, wherein the first valve includes a screw pump.

16. A method for disinfecting a plumbing fixture, the method comprising:
providing a predetermined volume of water to a mixing chamber;
providing a predetermined quantity of dry precursor to the mixing chamber;
opening an outlet valve; and
providing a flow of chlorine dioxide solution through the outlet valve to the plumbing fixture.

17. The method of claim 16, further comprising:
opening a water supply valve,
wherein the providing the predetermined volume of water to the mixing chamber includes providing the predetermined volume of water through the water supply valve.

18. The method of claim 16, further comprising:
opening a precursor valve,
wherein the providing the predetermined quantity of dry precursor to the mixing chamber includes providing the predetermined quantity of dry precursor through the precursor valve.

19. The method of claim 16, further comprising:
coupling a cartridge to the mixing chamber, the cartridge including the dry precursor.

20. The method of claim 16, wherein the plumbing fixture comprises one of a sink drain pipe, a shower drain pipe, a reservoir in a toilet, a urinal drain pipe, and a bathtub drain pipe.
